(19) United States
(12) Patent Application Publication
(10) Pub. No.: US 2015/0111970 A1
(43) Pub. Date: Apr. 23, 2015

(54) GENETIC MARKERS AND DIAGNOSTIC METHODS FOR RESISTANCE OF BREAST CANCER TO HORMONAL THERAPIES

(71) Applicant: Rutgers, The State University of New Jersey, New Brunswick, NJ (US)

(72) Inventors: Erhan Bilal, Somerset, NJ (US); Gyan Bhanot, Princeton, NJ (US); Shrider Ganesan, Westfield, NJ (US)

(21) Appl. No.: 14/521,202

(22) Filed: Oct. 22, 2014

Related U.S. Application Data
(62) Division of application No. 13/220,434, filed on Aug. 29, 2011, now abandoned.
(60) Provisional application No. 61/377,642, filed on Aug. 27, 2010.

(51) Int. Cl. C12Q 1/68; A61K 45/06; A61K 31/138

(52) U.S. Cl. CPC C12Q 1/6886; A61K 31/138; A61K 45/06; C12Q 2600/106; C12Q 2600/158; C12Q 2600/156; C12Q 2600/118
USPC 514/648

(57) ABSTRACT

This application provides a method to identify genetic markers associated with increased sensitivity or resistance to hormonal therapies using an outlier analysis. More specifically, this application discloses that amplifications on chromosomes 8 and 17 are associated with increased proliferation and poor outcome in ER-positive breast cancer, and amplicons 17q21.33-q25.1, 8p11.2 and 8q24.3 may be responsible for higher proliferation and poor outcome in the setting of antiestrogen, in particular Tamoxifen, treatment clinically observed in a subset of ER-positive, HER2-negative breast cancers. The invention also provides use of the identified genetic markers in the development of targeted treatments for antiestrogen-resistant ER-positive breast cancers as well as in improving current methods of drug response prediction.

### CROSS REFERENCE TO RELATED APPLICATIONS

The present application claims priority under 35 U.S.C. §119(e) to U.S. Provisional Patent Application Ser. No. 61/377,642, filed Aug. 27, 2010, the contents of which are hereby incorporated by reference in its entirety.

### FIELD OF THE INVENTION

This invention is related to identification of genetic markers for predicting or diagnosing resistance to hormonal therapies in patients with breast cancers. The invention identifies three amplified chromosomal regions, in particular the amplified loci on chromosomes 8 and 17, as the genetic markers for predicting and diagnosing antiestrogen (in particular Tamoxifen) resistant ER+ breast tumors. The inventions also provides use of these amplified chromosomal regions as target to develop therapeutic agents for the antiestrogen-resistant ER+ breast tumors.

### BACKGROUND OF THE INVENTION

Hormonal therapies, especially antiestrogens, have been widely used for treatment of breast cancers. Tamoxifen is one of the most frequently prescribed drugs for the treatment of ER-positive breast cancers with beneficial effects for a large number of patients. It is a well tolerated drug with low toxicity and has dramatically affected overall survival of women with ER-positive breast cancers (Clark, G. M. and McGuire, W. L., Seminars in Oncology, 1988, 15(2 Suppl. 1):20-5). Although hormonal therapy has dramatically improved the outcome of ER-positive breast cancers, there is still a significant subset of ER-positive breast cancer swho suffer early distant relapse despite endocrine therapy. This suggests that a subset of ER-positive breast cancers have intrinsic resistance to hormone therapy or there are additional mechanisms for tumor progression independent of the estrogen pathway.

Amplification of chromosomal region 17q12 harbouring ERBB2 (HER2) oncogene has been demonstrated to be associated with endocrine resistance in ER-positive breast cancers, with ER-positive/HER2-positive tumors having relatively poor outcome with hormonal treatment alone. However HER2 amplification does not account for all endocrine resistance, and there remains a considerable subset of ER-positive/HER2-negative tumors that suffer early relapse with endocrine therapy. These tumors tend to have high grade, high proliferative indices and high Oncotype DX recurrence scores, but the mechanism behind endocrine resistance in these poor prognosis ER-positive/HER2-negative tumors remains uncertain.

Therefore, better understanding of the biological mechanisms associated with Tamoxifen resistance are of considerable clinical significance and may provide new strategies in managing treatments for breast cancer patients.

### SUMMARY OF THE INVENTION

The present invention provides new insight into the mechanisms underlying resistance to hormone therapies in ER-positive breast cancers by analyzing three different cohorts of published gene expression data on early stage ER-positive breast cancers treated with Tamoxifen, which represents data from 268 ER-positive breast cancers. Outlier analysis was used to identify pathways and potential amplicons that were associated with poor outcome.

Thus, in one aspect the present invention provides a method of identifying a genetic marker associated with increased sensitivity or resistance to a hormonal therapy in treatment of a cancer, the method comprising: (1) collecting samples of gene expression data from a statistically significant number of patients having the cancer under a hormonal therapy; (2) monitoring and collecting data on the patients' responses to the hormonal therapy; (3) correlating the gene expression data of the samples with the patients' responses to the hormonal therapy; and (4) conducting an outlier analysis on the correlation between the gene expression data with the patients' responses to the hormonal therapy.

In another aspect the present invention provides a method of predicting or diagnosing resistance of a breast cancer in a patient to a hormonal therapy, the method comprising an assay on expression of a cell-cycle gene or an assay on enrichment of an amplified chromosomal region of the patient, wherein the amplified chromosomal region is a locus on chromosomes 8 and 17, and wherein over-expression of the cell-cycle gene or enrichment of the amplification of the chromosomal region indicates possible resistance of the patient's breast cancer to the hormonal therapy.

In another aspect the present invention provides use of an amplified chromosomal region selected from the group consisting of 17q12, 17q21.33-q25.1, 8p11.2 and 8q24.3 as a target or genetic marker to develop therapeutic agents for treatment of patients having an ER-positive breast cancer resistant to hormonal therapies.

Therefore, the present invention can lead to an assay which would identify breast cancer patients likely to have early recurrence under standard therapy. Such patients may benefit from additional chemotherapy. The assay would complement Oncotype Dx, which is currently in clinical use but does not address the risk factors identified by the discovery of the present inventors.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates PCA plots of over-expressed outliers. FIG. 1B illustrates PCA plots of under-expressed outliers. Outlier profiles of genes associated with differential survival are organized in a binary matrix where 1 indicates the presence of an outlier. The figure represents the projection of each gene's outlier profile on the first two principal components of the corresponding matrix. Clusters associated with good prognosis are circled in blue while clusters associated with bad prognosis are circled in red.

FIG. 2 illustrates a clustergram of the correlation matrix between selected over-expressed genes associated with poor survival under Tamoxifen treatment. Calculating Phi coefficients for the distribution of high outliers between every two genes found to be associated with Tamoxifen resistance in Figure lA produces a correlation matrix. This figure shows the resulting heatmap of the hierarchical clustering (Pearson correlation distance, complete linkage) of this correlation matrix. Genes in the same pathway or chromosomal region are clustered together as marked.

FIG. 3 shows patients with cell cycle pathway activation show poor survival outcome, Kaplan-Meier curves of the samples enriched for over-expressed cell cycle genes versus the rest of samples that don't show this feature.

FIG. 4 shows patients with 17q12, 17q21.33-q25.1, 8p11.2 and 8q24.3 amplifications show poor survival outcome, Kaplan-Meier curves of the samples with the 4 amplicons versus samples that do not have any of the chromosomal amplifications.

FIG. 5 illustrates Oncotype DX scores. Oncotype DX scores calculated across all 3 data sets are shown as mean values with standard errors for each group of samples listed on the vertical axes.

### DETAILED DESCRIPTION OF THE INVENTION

Most ER-positive breast cancers are treated with hormonal therapy using agents such as Tamoxifen which disrupt the estrogen signalling pathway. However, not all ER-positive cases respond to this therapy, and a significant subset of ER-positive breast cancers suffer early relapse despite hormonal therapy. This invention aims to identify genetic markers associated with increased sensitivity or resistance to Tamoxifen using outlier analysis. The present inventors collected 268 ER-positive samples of gene expression data from three separate published studies on tamoxifen treated early stage ER-positive breast cancers for which clinical follow-up was available. Outlier analysis was used to identify genes associated with differential survival distributions using Kaplan-Meyer survival curves. Additional correlation analysis and PCA clustering were used to identify pathways and chromosomal regions associated with differential survival.

Outlier analysis was used to identify pathways and potential amplicons that were associated with poor outcome. Pathway analysis demonstrated that over-expression of a set of cell cycle genes correlated with poor survival. Analysis of putative amplicons showed that increased expression of genes from 17q21.33-q25.1, 17q12, 8p11.2 and 8q24.3 was associated with poor outcome. The 17q12 amplicon contains HER2 and has been shown to be associated with poor outcome in ER-positive breast cancers. The other amplicons were previously documented in breast cancer and associated with poor outcome and harbour putative oncogenes such as LSM1 and HSF1. Since most of the samples enriched in the cell cycle pathway also have at least one of the amplicons, this suggests the amplifications on chromosomes 8 and 17 are associated with increased proliferation and poor outcome in ER-positive breast cancer. In addition to this, a relative Oncotype DX score was calculated for samples using normalized expression levels and published weights. This analysis showed that high Oncotype DX™ scores are generated by tumors having either the HER2 amplicon, or one of the other amplicons on chromosomes 17 and 8. In contrast, low Oncotype DX™ scores were found only for tumors that do not exhibit any of the identified chromosomal aberrations.

Outlier expression values were identified for three separate gene expression datasets obtained from patients with breast cancer undergoing therapy with the estrogen blocker Tamoxifen. The resulting outlier profiles were matched across datasets for each gene and combined by associating distant metastasis recurrence times for each sample identified as an outlier. In consequence, for each gene we can associate survival curves for outlier samples and for non-outlier samples that can then be compared. We kept only genes that had a significant change in survival between the two Kaplan-Meier curves defined by the corresponding distribution of outliers between samples. The resulting set is further reduced by iteratively eliminating genes with outlier profiles that don't correlate with at least one other profile from another gene, resulting in sets of genes that are over-expressed or under-expressed in roughly the same set of samples. This process ensures that subsequent pathway enrichment analysis make sense while at the same time eliminating false positives.

The existing standard to assign risk for breast cancers is Oncotype Dx, which is based on 21 genes that measure ER, HER2 status and proliferation using qRT-PCR. The present inventors discovered that there are at least three genomic regions whose amplification is a risk factor for early recurrence and is not identified by Oncotype Dx. Patients with these chromosomal amplifications (amplicons) can be identified at diagnosis and may benefit from additional chemotherapy. In addition, an analysis of the genes in these amplicons using cell-line assays would identify driver genes responsible for the additional risk which would be targets for developing therapeutics.

Thus in one aspect, the present invention provides a method of identifying a genetic marker associated with increased sensitivity or resistance to a hormonal therapy in treatment of a cancer, the method comprising: (1) collecting samples of gene expression data from a statistically significant number of patients having the cancer under a hormonal therapy; (2) monitoring and collecting data on the patients' responses to the hormonal therapy; (3) correlating the gene expression data of the samples with the patients' responses to the hormonal therapy; and (4) conducting an outlier analysis on the correlation between the gene expression data with the patients' responses to the hormonal therapy.

In one embodiment of this aspect, observation of a consistent correlation between over-expression of a chromosomal amplification (amplicon) with low responses of the patients to the hormonal therapy or poor survival of the patients indicates that the amplicon can be used as a genetic marker associated with resistance of the cancer to the hormonal therapy.

In another embodiment of this aspect, said statistically significant number for the samples is at least 10.

In another embodiment of this aspect, said statistically significant number for the samples is at least 20.

In another embodiment of this aspect, said statistically significant number for the samples is at least 50.

In another embodiment of this aspect, said statistically significant number for the samples is at least 100.

In another embodiment of this aspect, said statistically significant number for the samples is at least 150.

In another embodiment of this aspect, said statistically significant number for the samples is at least 200.

In another embodiment of this aspect, said statistically significant number for the samples is at least 250.

In another embodiment of this aspect, the cancer is a breast cancer.

In another embodiment of this aspect, the cancer is an ER-positive breast cancer.

In another embodiment of this aspect, the cancer is an ER-positive, HER2-negative breast cancer.

In another embodiment of this aspect, the hormonal therapy comprises treatment with an antiestrogen agent.

In another embodiment of this aspect, the hormonal therapy comprises treatment with an antiestrogen agent selected from Afimoxifene, Arzoxifene, Bazedoxifene, Cyclofenil, Lasofoxifene, Ormeloxifene, Raloxifene, Tamoxifen, Toremifene, Clomifene, Mepitiostane, Nafoxidine, and Fulvestrant.

In another embodiment of this aspect, observation of a consistent correlation between over-expressed outliers with a high response or survival rate of the patients indicates existence of a genetic marker of sensitivity of the cancer to the hormonal therapy.

In another embodiment of this aspect, the cancer is an ER-positive breast cancer, the hormonal therapy comprises treatment with an antiestrogen agent, and the genetic marker of sensitivity is enrichment for pathways including development and cell adhesion or over-expression of immune response genes.

In another aspect the present invention provides a method of predicting or diagnosing resistance of a breast cancer in a patient to a hormonal therapy, the method comprising an assay on expression of a cell-cycle gene or an assay on enrichment of an amplified chromosomal region of the patient, wherein the amplified chromosomal region is a locus on chromosomes 8 and 17, and wherein over-expression of the cell-cycle gene or enrichment of the amplification of the chromosomal region indicates possible resistance of the patient's breast cancer to the hormonal therapy.

In one embodiment of this aspect, the amplified chromosomal region is selected from 17q12, 17q21.33-q25.1, 8p11.2, and 8q24.3.

In another embodiment of this aspect, the cell-cycle gene is selected from GSDML, GRB7, PSMD3, STARD3, ERBB2a, PHB, SLC35B1, RAD51C, SUPT4H1, CLTCa, ABC1, PTRH2, APPBP2, TRIM37, USP32, CYB561, CCDC44, PSMC5, KPNA2, PSMD12, ICT1, ATP5H, MRPS7, SAP30BP, ASH2L, SPFH2, LSM1a, PROSC, WHSC1L1, BRF2, DDHD2, ATP6V1H, UBE2V2, MRPL15, COPS5, TCEB1, FAM82B, UQCRB, POLR2K, ATP6V1C1, EBAG9, ENY2, YWHAZ, RAD21, SQLE, MRPL13, BOP1, C8orf30A, C8orf33, CYC1, SIAHBP1, EXOSC4, FBXL6, GPR172A, GRINA, HSFla, ZNF250, RPL8, SCRIB, SHARPIN, VPS28, and ZNF7.

In another embodiment of this aspect, the cancer is an ER-positive breast cancer.

In another embodiment of this aspect, the cancer is an ER-positive, HER2-negative breast cancer.

In another embodiment of this aspect, the hormonal therapy comprises treatment with an antiestrogen agent.

In another embodiment of this aspect, the hormonal therapy comprises treatment with an antiestrogen agent selected from Afimoxifene, Arzoxifene, Bazedoxifene, Cyclofenil, Lasofoxifene, Ormeloxifene, Raloxifene, Tamoxifen, Toremifene, Clomifene, Mepitiostane, Nafoxidine, and Fulvestrant.

In another embodiment of this aspect, the hormonal therapy comprises treatment with Tamoxifen.

In another embodiment of this aspect, the method further comprises measuring ER, HER2 status and proliferation using qRT-PCR to obtain an Oncotype DX™ score, wherein an increased expression of the amplicon in combination with a high Oncotype DX™ score indicates an enhanced likelihood of the patient to develop resistance to the hormonal therapy, and wherein a normal expression of the amplicon in combination with a low Oncotype DX™ indicates a low likelihood of the patient to develop resistance to the hormonal therapy.

In another aspect the present invention provides use of an amplified chromosomal region selected from the group consisting of 17q12, 17q21.33-q25.1, 8p11.2 and 8q24.3 as a target or genetic marker to develop therapeutic agents for treatment of patients having an ER-positive breast cancer resistant to hormonal therapies.

In one embodiment of this aspect, the hormonal therapy comprises treatment with an antiestrogen agent.

In another embodiment of this aspect, the hormonal therapy comprises treatment with an antiestrogen agent selected from Afimoxifene, Arzoxifene, Bazedoxifene, Cyclofenil, Lasofoxifene, Ormeloxifene, Raloxifene, Tamoxifen, Toremifene, Clomifene, Mepitiostane, Nafoxidine, and Fulvestrant.

In another embodiment of this aspect, the hormonal therapy comprises treatment with Tamoxifen.

In another embodiment of this aspect, the breast cancer is HER2 negative.

It would be apparent to a person skilled in the art that any of the above embodiments may be applicable to other types of breast cancers under different hormonal therapies. The knowledge will also be useful in determining whether a hormonal therapy should be combined with other chemotherapy regimens for breast cancer patients.

The following non-limiting examples illustrate certain aspects of the invention.

### EXAMPLES

### Example 1

### Gene Pathway Patterns Correlate with Tamoxifen Sensitivity

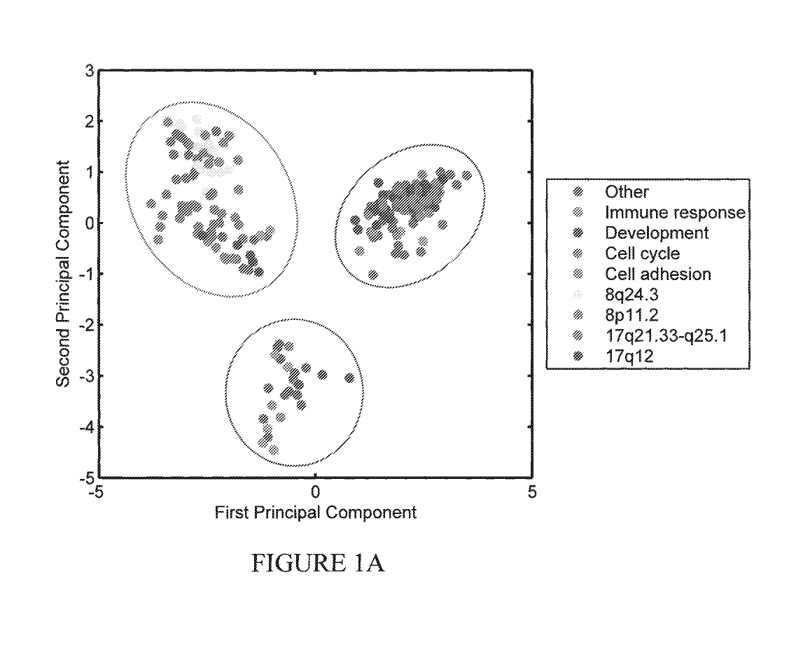

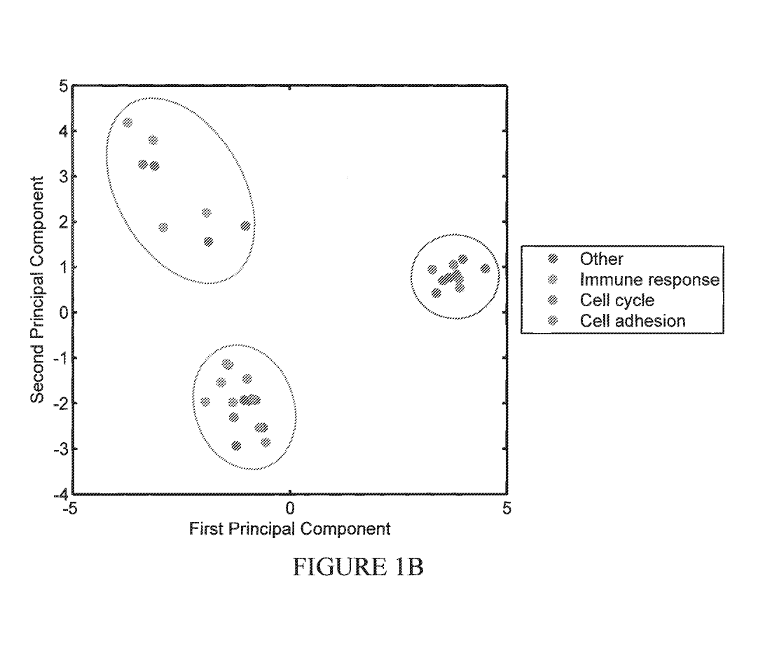

Outlier profiles of genes associated with differential survival are organized in a binary matrix where 1 indicates the presence of an outlier. FIG. 1 represents the projection of each gene's outlier profile on the first two principal components of the corresponding matrix for high outlier values (A) and respectively, low outlier values (B). The clusters circled in red are correlated with a poor outcome while the ones in blue have a better prognosis. This assignment was performed by examining survival for each individual gene outlier profile as listed in Additional File 1. Enrichment analysis over Gene Ontology (GO) annotations revealed that clusters are enriched with biological pathways and chromosomal regions presented in Table 1 (Nucleic Acids Research, 2008, 36(Database issue):D440-4). Enrichment was evaluated with a Fisher Exact test and a p-value <0.05 was used as a threshold.

TABLE 1
Gene patterns associated with Tamoxifen response
(Gene pathway enrichment analysis results).

| | Over-expression | Under-expression |
|---|---|---|
| Tamoxifen | Immune response | Cell cycle |
| sensitivity | Development | |
| | Cell adhesion | |
| Tamoxifen | Cell cycle | Immune response |
| resistance | Chr17q21.33-q25.1 | Cell adhesion |
| | Chr17q12 | |
| | Chr8p11.2 | |
| | Chr8q24.3 | |

Over-expressed outliers associated with good prognosis define two classes, one enriched for pathways including development and cell adhesion while the second one is described mostly by immune response genes. Most significantly we find over-expression of cell cycle genes in samples correlated with poor survival together with an enrichment of four chromosomal regions: 17q21.33-q25.1, 17q12, 8p11.2 and 8q24.3. The 17q12 amplicon contains HER2 and is known to be associated with relative resistance to hormonal therapy. Among over-expressed genes in 17q21.33-q25.1, 8p11.2 and 8q24.3 associated with poor survival outcome under Tamoxifen treatment we find cancer associated genes and putative oncogenes such as CLTC (Argani, P., et al., Oncogene, 2003, 22(34):5374-8; Patel, A. S., et al, Cancer Genetics and Cytogenetics, 2007, 176(2):107-14; De Paepe, P., et al., Blood, 2003, 102(7):2638-41), WHSC1L1, HSF1 (Dai, C., et al., Cell, 2007:1005-1018), LSM1 (Streicher K. L., et al., Oncogene, 2007:2104-2114) (Table 2).

TABLE 2
Over-expressed genes in chromosomal regions 17q12, 17q21.33-q25.1, 8p11.2
and 8q24.3 associated with Tamoxifen resistance (list of genes associated with
Tamoxifen resistance on chromosomes 8 and 17)

| Gene | Name | Cytoband |
|---|---|---|
| GSDML | gasdermin B | chr17q12 |
| GRB7 | growth factor receptor-bound protein 7 | chr17q12 |
| PSMD3 | proteasome (prosome, macropain) 26S subunit, | chr17q12 |
| | non-ATPase, 3 | |
| STARD3 | StAR-related lipid transfer (START) domain | chr17q12 |
| | containing 3 | |
| ERBB2^{a} | v-erb-b2 erythroblastic leukemia viral | chr17q12 |
| | oncogene homolog 2, neuro/glioblastoma | |
| | derived oncogene homolog (avian) | |
| PHB | prohibitin | chr17q21.33 |
| SLC35B1 | solute carrier family 35, member B1 | chr17q21.33 |
| RAD51C | RAD51 homolog C (S. cerevisiae) | chr17q22 |
| SUPT4H1 | suppressor of Ty 4 homolog 1 (S. cerevisiae) | chr17q22 |
| CLTC^{a} | clathrin, heavy chain (Hc) | chr17q23.1 |
| ABC1 | ATP-binding cassette, sub-family A | chr17q23.1 |
| | (ABC1), member 1 | |
| PTRH2 | peptidyl-tRNA hydrolase 2 | chr17q23.1 |
| APPBP2 | amyloid beta precursor protein | chr17q23.2 |
| | (cytoplasmic tail) binding protein 2 | |
| TRIM37 | tripartite motif-containing 37 | chr17q23.2 |
| USP32 | ubiquitin specific peptidase 32 | chr17q23.2 |
| CYB561 | cytochrome b-561 | chr17q23.3 |
| CCDC44 | coiled-coil domain containing 44 | chr17q23.3 |
| PSMC5 | proteasome (prosome, macropain) 26S subunit, | chr17q23.3 |
| | ATPase, 5 | |
| KPNA2 | karyopherin alpha 2 (RAG cohort 1, importin alpha 1) | chr17q24.2 |
| PSMD12 | proteasome (prosome, macropain) 26S subunit, | chr17q24.2 |
| | non-ATPase, 12 | |
| ICT1 | immature colon carcinoma transcript 1 | chr17q25.1 |
| ATP5H | ATP synthase, H+ transporting, | chr17q25.1 |
| | mitochondrial F0 complex, subunit d | |
| MRPS7 | mitochondrial ribosomal protein S7 | chr17q25.1 |
| SAP30BP | SAP30 binding protein | chr17q25.1 |
| ASH2L | ash2 (absent, small, or homeotic)-like | chr8p11.2 |
| | (Drosophila) | |
| SPFH2 | ER lipid raft associated 2 | chr8p11.2 |
| LSM1^{a} | LSM1 homolog, U6 small nuclear RNA | chr8p11.2 |
| | associated (S. cerevisiae) | |
| PROSC | proline synthetase co-transcribed | chr8p11.2 |
| | homolog (bacterial) | |
| WHSC1L1 | Wolf-Hirschhorn syndrome candidate 1-like 1 | chr8p11.2 |
| BRF2 | BRF2, subunit of RNA polymerase III | chr8p12 |
| | transcription initiation factor, BRF1-like | |
| DDHD2 | DDHD domain containing 2 | chr8p12 |
| ATP6V1H | ATPase, H+ transporting, lysosomal | chr8q11.2 |
| | 50/57 kDa, V1 subunit H | |
| UBE2V2 | ubiquitin-conjugating enzyme E2 variant 2 | chr8q11.21 |
| MRPL15 | mitochondrial ribosomal protein L15 | chr8q11.23 |
| COPS5 | COP9 constitutive photomorphogenic | chr8q13.2 |
| | homolog subunit 5 (Arabidopsis) | |
| TCEB1 | transcription elongation factor B (SIII), | chr8q21.11 |
| | polypeptide 1 (15 kDa, elongin C) | |
| FAM82B | family with sequence similarity 82, | chr8q21.3 |
| | member B | |
| UQCRB | ubiquinol-cytochrome c reductase | chr8q22 |
| | binding protein | |
| POLR2K | polymerase (RNA) II (DNA directed) | chr8q22.2 |
| | polypeptide K, 7.0 kDa | |
| ATP6V1C1 | ATPase, H+ transporting, lysosomal | chr8q22.3 |
| | 42 kDa, V1 subunit C1 | |
| EBAG9 | estrogen receptor binding site associated, | chr8q23 |
| | antigen, 9 | |
| ENY2 | enhancer of yellow 2 homolog | chr8q23.1 |
| | (Drosophila) | |
| YWHAZ | tyrosine 3-monooxygenase/tryptophan 5- | chr8q23.1 |
| | monooxygenase activation protein, zeta | |
| | polypeptide | |
| RAD21 | RAD21 homolog (S. pombe) | chr8q24 |
| SQLE | squalene epoxidase | chr8q24.1 |
| MRPL13 | mitochondrial ribosomal protein L13 | chr8q24.12 |
| BOP1 | block of proliferation 1 | chr8q24.3 |
| C8orf30A | chromosome 8 open reading frame 30A | chr8q24.3 |
| C8orf33 | chromosome 8 open reading frame 33 | chr8q24.3 |
| CYC1 | cytochrome c-1 | chr8q24.3 |
| SIAHBP1 | poly-U binding splicing factor 60 KDa | chr8q24.3 |
| EXOSC4 | exosome component 4 | chr8q24.3 |
| FBXL6 | F-box and leucine-rich repeat protein 6 | chr8q24.3 |
| GPR172A | G protein-coupled receptor 172A | chr8q24.3 |
| GRINA | glutamate receptor, ionotropic, N-methyl | chr8q24.3 |
| | D-aspartate-associated protein 1 | |
| | (glutamate binding) | |
| HSF1^{a} | heat shock transcription factor 1 | chr8q24.3 |
| ZNF250 | In multiple Geneids | chr8q24.3 |
| RPL8 | ribosomal protein L8 | chr8q24.3 |
| SCRIB | scribbled homolog (Drosophila) | chr8q24.3 |
| SHARPIN | SHANK-associated RH domain | chr8q24.3 |
| | interactor | |
| VPS28 | vacuolar protein sorting 28 homolog (S. cerevisiae) | chr8q24.3 |
| ZNF7 | zinc finger protein 7 | chr8q24.3 |

^{a}Cancer related genes (CLTC) and oncogenes (ERBB2, LSM1 and HSF1).

For under-expressed outliers with good prognosis we find enrichment of the cell cycle pathway, while the immune response and cell adhesion are associated with poor prognosis. This inverse relationship confirms the strong association of these pathways with prognosis in ER-positive breast cancers.

### Example 2

### Multiple Chromosomal Amplifications Associated with High Grade, Tamoxifen Resistant Breast Tumors

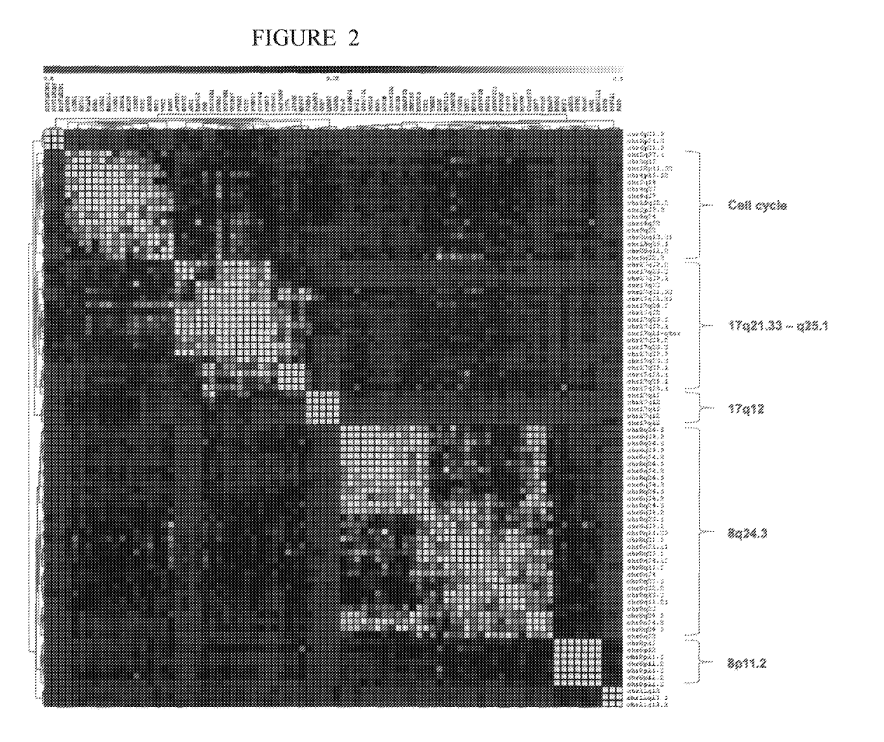

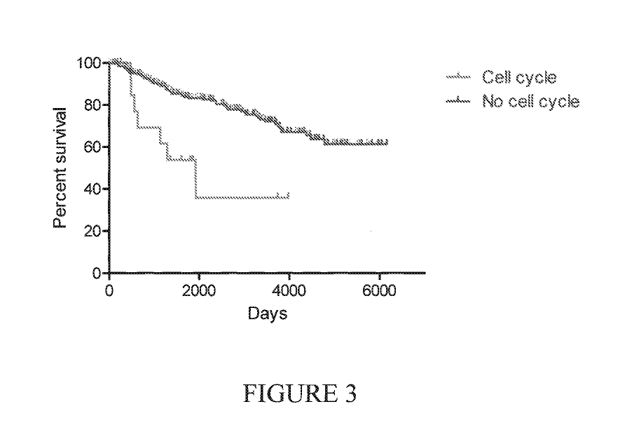

Oncogenes found in Table 2 are part of known amplified chromosomal regions also listed in Table 1 as gene patterns associated with poor prognosis. We focused on these patterns by clustering the corresponding correlation matrix. This is displayed as a heatmap in FIG. 2 where we can observe that genes from the same pathway/region tend to be more correlated with each other than the rest. The cell cycle pathway correlates partly with all the amplicons, suggesting that any of these amplicons is associated with increased expression of cell cycle pathway genes. However each amplicon is poorly correlated with each other, unless they are on the same chromosome. These data suggests that the presence of each amplicon is functionally independent of each other and can potentially affect treatment response by amplifying selected oncogenes.

The association between enrichment of the cell cycle genes and the presence of putative amplicons, was further examined. Samples with enrichment of any of the four amplicons or the cell cycle pathway enrichment were identified by requiring at least 50% of gene markers in each group to be over-expressed, i.e. is marked as a high outlier in the respective sample. It was found that most samples (93%) that over-express cell cycle genes display at least one of the four chromosomal amplifications, further suggesting a causal relationship between the presence of these amplicons and tumor proliferation. By computing correlations between all 5 patterns found to be associated with Tamoxifen resistance (Table 3), in the sample space, we see that all amplicons are positively associated with the cell cycle group and with each other in the case of regions on the same chromosome.

TABLE 3
Sample correlations between gene patterns
associated with bad prognosis

| | cell cycle | 17q12 | 17q21.33-q25.1 | 8p11.2 | 8q24.3 |
|---|---|---|---|---|---|
| cell cycle | 1.00 | 0.21 | 0.26 | 0.20 | 0.22 |
| 17q12 | 0.21 | 1.00 | 0.18 | 0.01 | 0.00 |
| 17q21.33-q25.1 | 0.26 | 0.18 | 1.00 | 0.07 | 0.23 |
| 8p11.2 | 0.20 | 0.01 | 0.07 | 1.00 | 0.26 |
| 8q24.3 | 0.22 | 0.00 | 0.23 | 0.26 | 1.00 |

* Values represent Phi coefficients measuring the strength of association between the group of samples that over-express cell cycle genes and amplicons 17q12, 17q21.33-q25.1, 8p11.2 and 8q24.3

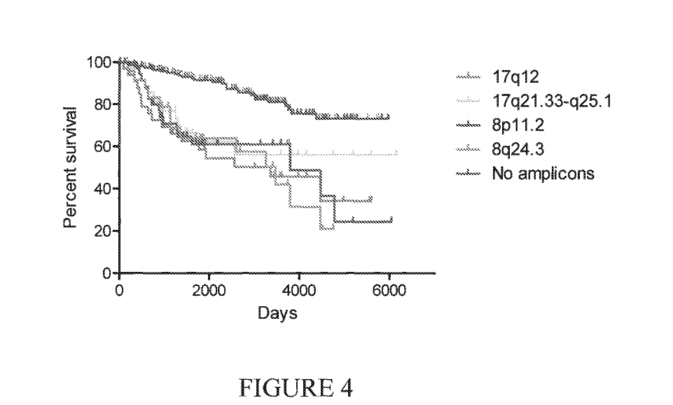

Other associations are presented in Table 4 where we can see that presence of 17q12, 8p11.2 and 8q24.3 enrichment is associated with high grade (p-value<0.05 computed with the Fisher Exact Test) while node status is not correlated with any of the amplicons. Presence of any of the four amplicons is associated with significantly decreased five year survival when compared with tumors that do not harbour any amplicons (FIG. 4). Similarly presence of the cell cycle pathway also is associated with significantly reduced survival when compared with tumors that lack this signature.

TABLE 4
Amplicon Properties*

| | | | | | High grade | Node |
|---|---|---|---|---|---|---|
| | Median | | | | tumor | status |
| | survival | Hazard | | Logrank | enrichment | association |
| Amplicon | (days) | ratio | 95% CI | p-value | p-value | p-value |
| 17q12 | 3355 | 4.0929 | 3.8397-21.9970 | <0.0001 | 0.0002 | 0.8523 |
| 17q21.33-q25.1 | — | 3.1402 | 2.1718-13.6229 | 0.0003 | 0.2057 | 0.8564 |
| 8p11.2 | 3795 | 3.7512 | 3.1784-18.3088 | <0.0001 | 0.0416 | 0.8311 |
| 8q243 | 3468 | 4.2870 | 4.3216-34.0834 | <0.0001 | 0.0020 | 0.8564 |

*Hazard ratio and logrank p-values are computed with reference to the set of samples that don't have any of the presented amplicons.

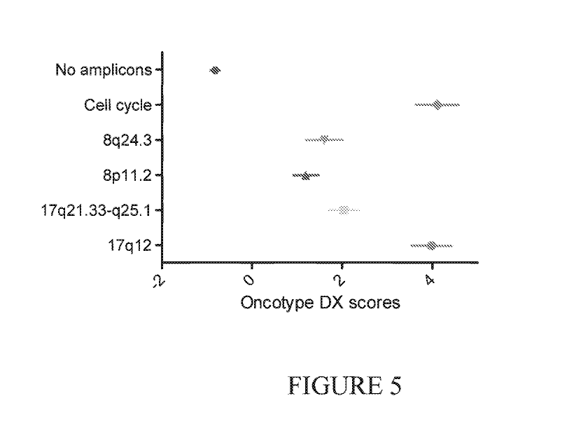

Another validated marker of poor outcome in ER-positive breast cancers with hormonal treatment is the Oncotype DX assay. This assay uses a linear combination of the expression of 21 genes to generate a single recurrence score. When the same gene panel is used to generate a relative Oncotype DX score (FIG. 5) using normalized expression levels and published weights (Paik, S., et al.; New Eng. J. Med., 2004, 351(27):2817-26), we found that the presence of any of these amplicons was associated with higher recurrence scores, while tumors lacking the amplicons had low recurrence score.

### Example 3

### Data Processing

Three gene expression data sets collected from breast cancer patients were obtained from the Gene Expression Omnibus website (GEO:www.ncbi.nlm.nih.gov/geo) accession number GSE6532 (Loi, S., et al., J. Clin. Oncol., 2007, 25(10):1239-46). The sets are abbreviated with KIT, OXFT and GUYT representing the institutions from where they were collected: Uppsala University Hospital, Uppsala, Sweden, John Radcliffe Hospital, Oxford, United Kingdom and Guys Hospital, London, United Kingdom. They comprise of 81, 109 and 87 ER-positive breast cancer samples from patients treated with the estrogen blocker Tamoxifen together with follow up disease progression information. The expression data were obtained on Affymetrix (Affymetrix, Santa Clara, Calif.) microarray platforms U133A/B (KIT & OXFT) and U133Plus2 (GUYT), then MASS normalized. In order to combine the three sets into one analysis, probes corresponding to genes that were not present across all samples were discarded. Multiple probes corresponding to the same gene were compressed to the one with the biggest median after taking log 2 of each intensity value.

### Example 4

### Outlier Analysis of Gene Expression Data Sets

For each gene, the expression values were median centered and then divided by the median absolute deviation (MAD) as described in Tomlins et al (Tomlins, S. A., et al., Science, 2005, 310(5748):644-8). Median and MAD were used here instead of the usual mean and standard deviation because they are less influenced by the presence of outliers. This step was performed separately for KIT, OXFT and GUYT data sets in order to avoid distribution biases that arise from the merger of separate expression array tables.

After normalization, outliers were separated in high/low groups, corresponding to samples with normalized values bigger/smaller than 90% and respectively 10% quantiles for each array. This result is organized, across all arrays and data sets, into two binary matrices, B₁ and B₂, corresponding to high and low outliers. For both matrices, B(i,j)=1 if gene i is found as an outlier in sample j while B(i,j)=0 for the rest.

In the next step, genes with less than 10 corresponding outliers across all samples were discarded since they weren't informative enough. For the rest, the distribution of outliers across the samples corresponds to an outlier profile which defines two classes for each gene: the class with aberrant expression of the corresponding gene and the rest of the samples where mRNA expressions are at normal levels as defined by the sample majority. We can then associate Kaplan-Meier curves to the two classes and assess differential survival between them with a log-rank test. The full list of genes together with the mentioned properties is listed in Additional File 1, which is an Excel 2003 file containing a table of outlier association results for all genes used in the analysis along with the outlier score, hazard ratio, corresponding p-value and logrank p-values, as disclosed in Provisional Application No. 61/377,642, which is hereby incorporated by reference.

### Example 3

### Identification of Predictive Gene Patterns for Tamoxifen Sensitivity

In order to perform any kind of pathway enrichment analysis on the gene set, previously found to be associated with good or poor survival, genes need to have a similar outlier profile, which means they need to be over/under-expressed in roughly the same samples. This corresponds to tightly correlated genes in the binary space of matrices B₁ and B₂. One suitable correlation measure is the Phi coefficient which is equivalent to a Pearson correlation between the rows of matrices B₁ and B₂. Let C₁ and C₂ be the covariance matrices between the rows of B1 and B₂ respectively, then R_{1,2}(i,j)=C_{1,2}(i,j)/\sqrt{C_{1,2}(i,i)C_{1,2}(j,j)} is the matrix of correlation coefficients between the outlier profiles of the genes in B_{1,2}.

Clusters of tightly correlated genes were identified by iteratively removing row i and column j with R_{1,2}(i,j)<0.5 until a stable set was obtained, meaning the size of the reduced matrix R′ stops changing. PCA plots of the resulting reduced matrices B₁ and B₂ identify distinct groups of highly correlated genes that are now suitable for pathway enrichment analysis. Gene clusters in FIGS. 1A and 1B are associated with bad/good prognosis based on the survival profiles defined by the genes within each cluster. Further, each gene is labelled with the appropriate pathway information taken from the Gene Ontology² database together with chromosomal location information obtained from Affymetrix annotation files. Fisher Exact test was used to assess the significance of pathways and chromosomal location enrichment for each group of genes defined by the clusters in FIGS. 1A and 1B.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention which is defined by the following claims.

1-20. (canceled)

21. A method of treating breast cancer in a patient comprising
performing an assay on expression of a cell-cycle gene;
performing and an assay on enrichment of a locus on chromosomes 8 and 17;
identifying the patient with breast cancer having over-expression of the cell-cycle gene and the enrichment of at least one locus on chromosomes 8 and 17;
administering to the patient an antiestrogen agent and a chemotherapeutic agent.

22. A method of treating breast cancer in a patient comprising
performing an assay on expression of a cell-cycle gene;
performing and an assay on enrichment of a locus on chromosomes 8 and 17;
identifying the patient with breast cancer having low to normal expression of the cell-cycle gene and minimal enrichment of a locus on chromosomes 8 and 17;
administering to the patient an antiestrogen agent.
